(19) United States
(12) Patent Application Publication
(10) Pub. No.: US 2024/0117297 A1
(43) Pub. Date: Apr. 11, 2024

(54) PARA-AMINOBENZOIC ACID-PRODUCING MICROORGANISM

(71) Applicant: INDUSTRIAL TECHNOLOGY RESEARCH INSTITUTE, Hsinchu (TW)

(72) Inventors: Pei-Ching CHANG, Zhubei City (TW); Jhong-De LIN, Kaohsiung City (TW); Ya-Lin LIN, Hsinchu City (TW); Hung-Yu LIAO, Taoyuan City (TW); Hsiang Yuan CHU, Hsinchu City (TW); Jie-Len HUANG, Hsinchu City (TW)

(73) Assignee: INDUSTRIAL TECHNOLOGY RESEARCH INSTITUTE, Hsinchu (TW)

(21) Appl. No.: 18/148,112

(22) Filed: Dec. 29, 2022

(30) Foreign Application Priority Data
Oct. 6, 2022 (TW) 111137976

(51) Int. Cl. C12N 1/20

(52) U.S. Cl. CPC C12N 1/205; C12R 2001/19

(57) ABSTRACT

A p-aminobenzoic acid-producing microorganism is provided. The p-aminobenzoic acid-producing microorganism is obtained by a method for preparing a p-aminobenzoic acid-producing microorganism. The method for preparing a p-aminobenzoic acid-producing microorganism includes (a) performing an acclimation process on a source microorganism with at least one sulfonamide antibiotic to obtain at least one acclimatized microorganism and (b) screening out at least one p-aminobenzoic acid-producing microorganism from the at least one acclimatized microorganism, wherein the at least one p-aminobenzoic acid-producing microorganism has a higher p-aminobenzoic acid titer than the source microorganism.

### CROSS REFERENCE TO RELATED APPLICATION

The present application is based on, and claims priority from, Taiwan Application Serial Number 111137976, filed Oct. 6, 2022, the disclosure of which is hereby incorporated by reference herein in its entirety.

### REFERENCE TO AN ELECTRONIC SEQUENCE LISTING

The contents of the electronic sequence listing (9044C-A28636-US ST26 sequence listing. xml; Size: 16,638 bytes; and Date of Creation: Nov. 21, 2022) is herein incorporated by reference in its entirety.

### TECHNICAL FIELD

The present disclosure relates to the biosynthesis of para (p)-aminobenzoic acid (PABA), in particular to microorganisms which produce p-aminobenzoic acid.

### BACKGROUND

Para (p)-Aminobenzoic acid (PABA) is a representative monomer of key high-value aromatics for industrial use, and derivatives thereof can be widely used in fields of textiles, plastic components, electronics, health food, cosmetics and the like.

Traditionally, p-aminobenzoic acid monomer is obtained by chemical synthesis. However, since the chemical synthesis of p-aminobenzoic acid monomer requires several synthesis steps using strong acid, high temperature and high pressure, heavy metals and the like, and the synthesis process has the risk of explosion, it is almost impossible to continue to develop under the current environmental protection regulations of various countries.

Therefore, there is an urgent need for a novel and safe and non-toxic preparation method of p-aminobenzoic acid.

### SUMMARY

The present disclosure provides a p-aminobenzoic acid (PABA)-producing microorganism. The p-aminobenzoic acid-producing microorganism is obtained by a method for preparing a p-aminobenzoic acid-producing microorganism. The method for preparing a p-aminobenzoic acid-producing microorganism comprises (a) performing an acclimation process on a source microorganism with at least one sulfonamide antibiotic to obtain at least one acclimatized microorganism, and (b) screening out at least one p-aminobenzoic acid-producing microorganism from the at least one acclimatized microorganism, wherein the at least one p-aminobenzoic acid-producing microorganism has a higher p-aminobenzoic acid than the source microorganism.

Moreover, the present disclosure also provides another p-aminobenzoic acid-producing microorganism. The p-aminobenzoic acid-producing microorganism is derived from a source microorganism, wherein compared to the source microorganism, the p-aminobenzoic acid-producing microorganism has: overexpression of a gene related to citrate and malate biosynthesis; and/or suppressive expression of a gene related to glutamine metabolism, and wherein the at least one p-aminobenzoic acid-producing microorganism has a higher p-aminobenzoic acid titer than the source microorganism.

In addition, the present disclosure also provides a p-aminobenzoic acid-producing Escherichia coli, which is deposited at Leibniz Institute DSMZ-German Collection of Microorganisms and Cell Cultures and whose deposit number is DSM 34363.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 is a schematic diagram of the general biosynthetic pathway of folate in microorganisms.

FIG. 2A shows the design of plasmid ITRIWP1.

FIG. 2B shows the design of plasmid ITRIWP2.

FIG. 2C shows the design of plasmid ITRIWP3.

FIG. 3A shows a HPLC chromatogram of a standard of p-aminobenzoic acid.

FIG. 3B shows a standard curve of p-aminobenzoic acid.

FIG. 4 shows the growth rates of Escherichia coli BW1, BW2 and BW3 at 0 or 5 mg/L sulfanilamide in one embodiment.

FIG. 5 shows the growth rates of Escherichia coli JY1, JY2 and JY3 and Escherichia coli BW at 50, 100 or 1000 mg/L sulfanilamide in one embodiment.

FIG. 6 shows the growth rates of Escherichia coli JY31, JY32 and JY33 and Escherichia coli BW at 1000, 1500, 2000, 2500 or 3000 mg/L sulfanilamide in one embodiment.

FIG. 7 shows the titers of p-aminobenzoic acid of Escherichia coli JY33P1B, JY33P2B, JY33P3B, JY33P4B and JY33P5B and Escherichia coli BW in 24 and 48 hours in one embodiment, respectively.

FIG. 8 shows the biosynthetic pathway of the p-aminobenzoic acid of Escherichia coli JY33P5B in one embodiment.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

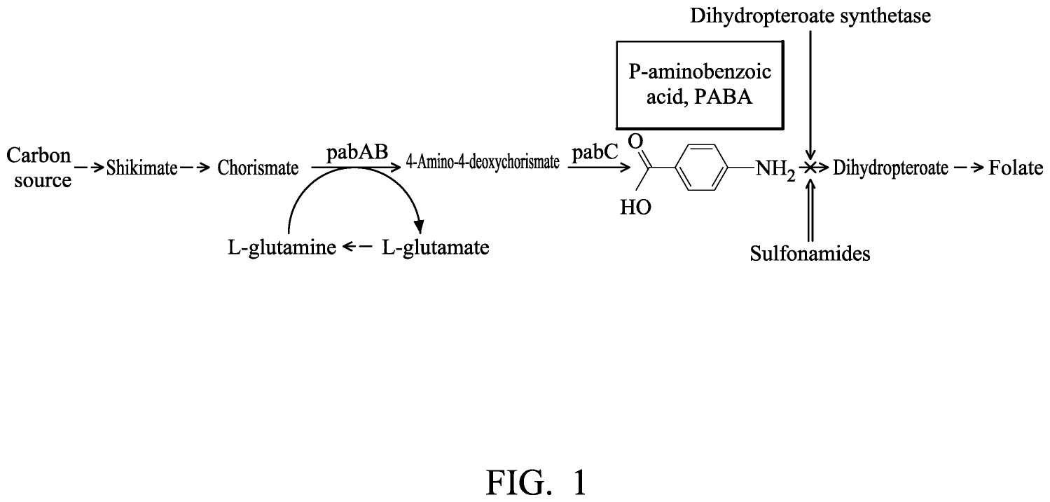

Folate is necessary for the survival of microorganisms. When a microorganism is unable to synthesize folate, the microorganism will die. FIG. 1 is a schematic diagram of the general biosynthetic pathway of folate in microorganisms.

Please refer to FIG. 1. In the folate biosynthetic pathway in microorganisms, p-aminobenzoic acid (PABA) is the intermediate product produced by 4-amino-4-deoxychorismate through catalysis, and p-aminobenzoic acid can be catalyzed by dihydropteroate synthetase to form dihydropteroate, and dihydropteroate can be further catalyzed by follow-up other downstream enzymes in the folate biosynthetic pathway to finally form folate.

Sulfonamide antibiotics can compete for the combination of p-aminobenzoic acid with dihydropteroate synthase to make microorganisms unable to fully produce dihydropteroate required for the subsequent synthesis of folate in the folate biosynthetic pathway, so that folate cannot be fully synthesized, eventually leading to the death of microorganisms.

On the other hand, the use of sulfonamide antibiotics can result in the accumulation of p-aminobenzoic acid in microorganisms due to blocking and/or inhibiting the pathway of p-aminobenzoic acid forming dihydropteroate through catalysis by dihydropteroate synthase. Namely, when a microorganism is acclimatized by sulfonamide antibiotics and can survive by synthesizing folate through other pathways, the acclimatized microorganism should have higher titer of p-aminobenzoic acid than before acclimation.

The term “titer” used herein is the concentration of biosynthetic substance produced by microorganism.

According to the foregoing, the present disclosure develops and provides a novel method for preparing a p-aminobenzoic acid-producing microorganism. In the method for preparing a p-aminobenzoic acid-producing microorganism of the present disclosure mentioned above, the use of sulfonamide antibiotics can drive the evolution of metabolic pathways of microorganisms and/or affect the expression of specific genes of microorganisms, thereby obtaining microorganisms with high p-aminobenzoic acid titer.

Specifically, the method for preparing a p-aminobenzoic acid-producing microorganism of the present disclosure may comprise, but is not limited to, the following steps.

First, an acclimation process is performed on a source microorganism with at least one sulfonamide antibiotic to obtain at least one acclimatized microorganism.

Next, at least one p-aminobenzoic acid-producing microorganism is screened out from the at least one acclimatized microorganism mentioned above.

Compared to the source microorganism mentioned above, the obtained at least one p-aminobenzoic acid-producing microorganism mentioned above may have a higher p-aminobenzoic acid titer.

The use concentration of at least one sulfonamide antibiotic mentioned above may be about 10-5000 mg/L, such as about 20-4500 mg/L, about 30-4000 mg/L, about 40-3500 mg/L, about 50-3000 mg/L, about 10-3000 mg/L, about 20-2500 mg/L, about 30-2000 mg/L, about 40-1500 mg/L, about 50-1000 mg/L, about 250-5000 mg/L, about 500-4500 mg/L, about 750-4000 mg/L, about 800-3500 mg/L, about 1000-3000 mg/L, about 50 mg/L, about 100 mg/L, about 1000 mg/L, about 1500 mg/L, about 2000 mg/L, about 2500 mg/L, about 3000 mg/L, etc., but it is not limited thereto. In one embodiment, the use concentration of at least one sulfonamide antibiotic mentioned above may be about 50-1000 mg/L. In another embodiment, the use concentration of at least one sulfonamide antibiotic mentioned above may be about 1000-3000 mg/L.

The at least one sulfonamide antibiotic mentioned above may comprise, but is not limited to, sulfanilamide, sulfacetamide, sulfadiazine, sulfasalazine, sulfadimidine, sulfafurazole, etc., or any combination thereof. In one embodiment, the at least one sulfonamide antibiotic mentioned above may be sulfanilamide.

Moreover, based on the foregoing, it is known that the folate biosynthetic pathway of the microorganisms mentioned above originally exists in general microorganisms. In other words, the source microorganism mentioned above in the method for preparing a p-aminobenzoic acid-producing microorganism of the present disclosure may be a microorganism without any genetic modification, or may also be a genetically modified microorganism, as long as the general biosynthetic pathway of folate in microorganisms mentioned above (referring FIG. 1) exist in the microorganism. In one embodiment, the source microorganism mentioned above may be a genetically modified microorganism.

The genetically modified microorganism mentioned above may contain at least one exogenous gene, but it is not limited thereto. The at least one exogenous gene contained in the genetically modified microorganism mentioned above may comprise at least one of pabA, pabB, pabC, etc., but is not limited thereto. In one embodiment, the at least one exogenous gene contained in the genetically modified microorganism mentioned above may comprise, but is not limited to, pabA, pabB and pabC.

There is no particular limitation on the form in which the at least one exogenous gene contained in the foregoing genetically modified microorganism is preset in the foregoing genetically modified microorganism, as long as the at least one of the exogenous genes mentioned above can be expressed in this genetically modified microorganism and does not negatively affect other endogenous genes. In one embodiment, the at least one exogenous gene mentioned above may be present in at least one vector contained in the genetically modified microorganism mentioned above, and example of the vector mentioned above may comprise, but is not limited to, a plasmid or a viral vector. In another embodiment, the at least one exogenous gene mentioned above may be inserted into the chromosomal DNA of the genetically modified microorganism mentioned above.

Furthermore, example of the source microorganism mentioned above may comprise bacteria belonging to the genus Escherichia, bacteria belonging to the genus Corynebacterium, bacteria belonging to the genus Bacillus, bacteria belonging to the genus Pseudomonas, yeast belonging to the genus Yarrowia, yeast belonging to the genus Saccharomyces or yeast belonging to the genus Pichia, but it is not limited thereto.

Example of bacteria belonging to the genus Escherichia may comprise, but is not limited to, Escherichia coli. Moreover, the foregoing Escherichia coli may comprise, but is not limited to, Escherichia coli BW25113, K12, DH5a, BL21, XL1-blue and the like. Example of bacteria belonging to the genus Corynebacterium may comprise, but is not limited to, Corynebacterium glutamicum. Example of bacteria belonging to the genus Bacillus may comprise, Bacillus subtilis, but it is not limited to. Example of bacteria belonging to the genus Pseudomonas may comprise, but is not limited to, Pseudomonas putida. Furthermore, the yeast belonging to the genus Yarrowia may comprise Yarrowia lipolytica, but it is not limited thereto. Yeasts belonging to the genus Saccharomyces may comprise, but is not limited to, Saccharomyces cerevisiae. In addition, example of yeast belonging to the genus Pichia may comprise, Pichia pastoris, but it is not limited thereto.

In one embodiment, the source microorganism mentioned above may comprise Escherichia coli, Corynebacterium glutamicum, Bacillus subtilis, Pseudomonas putida, Yarrowia lipolytica, Saccharomyces cerevisiae, Pichia pastoris, etc., but is not limited thereto.

In another embodiment, the source microorganism mentioned above may be Escherichia coli. Moreover, in one specific embodiment, the source microorganism mentioned above may be a genetically modified Escherichia coli.

In addition, in the foregoing specific embodiment in which the source microorganism mentioned above may be a genetically modified Escherichia coli, the genetically modified Escherichia coli mentioned above may contain at least one exogenous gene, but it is not limited thereto. The at least one exogenous gene contained in the genetically modified Escherichia coli mentioned above may comprise, but is not limited to, at least one of pabA, pabB and pabC, etc. In one embodiment, the at least one exogenous gene contained in the genetically modified Escherichia coli mentioned above may comprise, but is not limited to, pabA, pabB and pabC. Moreover, in the foregoing specific embodiment in which the source microorganism mentioned above can be a genetically modified Escherichia coli, the genetically modified Escherichia coli may be genetically modified Escherichia coli BW25113.

In one embodiment, in the method for preparing a p-aminobenzoic acid-producing microorganism of the present disclosure, the foregoing acclimation process performed on a source microorganism with at least one sulfonamide antibiotic may comprise at least one acclimation culture, but is not limited thereto. Namely, the foregoing acclimation process performed on a source microorganism with at least one sulfonamide antibiotic may have only one acclimation culture or have two or more acclimation cultures. Number of acclimation culture is not particularly limited, and may be determined according to the acclimation circumstances of the source microorganism, but it is not limited thereto. When foregoing acclimation process performed on a source microorganism with at least one sulfonamide antibiotic has two or more acclimation cultures, the type of at least one sulfonamide antibiotic used in each acclimation culture can be the same or can be different. Similarly, when foregoing acclimation process performed on a source microorganism with at least one sulfonamide antibiotic has two or more acclimation culture, the concentration of at least one sulfonamide antibiotic used in each acclimation culture can be the same or can be different. In one embodiment, the concentration of at least one sulfonamide antibiotic used in each acclimation culture may be gradually increased. In another embodiment, the concentration of at least one sulfonamide antibiotic used in each acclimation culture may be gradually decreased.

Moreover, in one embodiment, the at least one acclimation culture mentioned above may comprise a first acclimation culture, but it is not limited thereto.

In the first acclimation culture mentioned above, the source microorganism mentioned above may be cultured in the presence of the at least one sulfonamide antibiotic mentioned above at a first concentration to obtain at least one first acclimatized microorganism.

In the first acclimation culture mentioned above, the at least one sulfonamide antibiotic mentioned above may comprise a first sulfonamide antibiotic, but it is not limited thereto. The first sulfonamide antibiotic mentioned above may comprise, but is not limited to, sulfanilamide, sulfacetamide, sulfadiazine, sulfasalazine, sulfadimidine, sulfafurazole, etc., or any combination thereof. In one embodiment, the first sulfonamide antibiotic mentioned above may be sulfanilamide.

In the first acclimation culture mentioned above, the first concentration of the at least one sulfonamide antibiotic mentioned above may be about 10-3000 mg/L, such as about 20-2500 mg/L, about 30-2000 mg/L, about 40-1500 mg/L, about 50-1000 mg/L, about 10 mg/L, about 15 mg/L, about 20 mg/L, about 25 mg/L, about 30 mg/L, about 40 mg/L, about 50 mg/L, about 60 mg/L, about 70 mg/L, about 80 mg/L, about 90 mg/L, about 100 mg/L, about 200 mg/L, about 250 mg/L, about 300 mg/L, about 400 mg/L, about 500 mg/L, about 600 mg/L, about 700 mg/L, about 800 mg/L, about 900 mg/L, about 1000 mg/L, but it is not limited thereto.

Furthermore, the culturing time for the first acclimation culture is not particularly limited, depending on the type of microorganism cultured, the culture method (such as batch culture and continuous culture), external environment (such as external environment temperature, external environment humidity and culture container/tank size), culture conditions (such as culture temperature, culture volume, composition of culture medium, type of culture medium, pH value of the culture environment, stirring rate and aeration rate), microbial growth circumstances, etc., or any combination thereof. In one embodiment, the culturing time for the first acclimation culture mentioned above may be about 6-72 hours, such as about 12-66 hours, about 18-60 hours, about 24-54 hours, about 30-48 hours, about 12-48 hours, about 6 hours, about 12 hours, about 18 hours, about 24 hours, about 30 hours, about 48 hours, about 54 hours, about 60 hours, about 66 hours and about 72 hours, but it is not limited thereto. In one specific embodiment, the culturing time for the first acclimation culture mentioned above may be about 24 hours.

Moreover, similarly, the culture temperature for the first acclimation culture mentioned above is not particularly limited and may depend on the type of microorganism cultured, the culture method (such as batch culture and continuous culture), external environment (such as external environment temperature, external environment humidity and culture container/tank size), culture conditions (such as culture time, culture volume, composition of culture medium, type of culture medium, pH value of the culture environment, stirring rate and aeration rate), microbial growth circumstances, etc., or any combination thereof. In one embodiment, the culture temperature for the first acclimation culture mentioned above may be about 20-45° C., such as about 22-44° C., about 25-43° C., about 27-44° C., about 30-40° C., about 32-38° C., about 35-37° C., about 22-35° C., about 24-32° C., about 25-30° C., about 25° C., about 27° C., about 28° C., about 29° C., about 30° C., about 32° C., about 33° C., about 34° C., about 35° C., about 36° C., about 37° C., about 37.5° C., about 38° C., about 39° C., about 40° C., about 42° C. and about 45° C., but it is not limited thereto. In one specific embodiment, the culture temperature for the first acclimation culture mentioned above may be about 37° C.

The pH value of the culture environment for the first acclimation culture mentioned above is not particularly limited, and may depend on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), microbial growth circumstances, etc., or any combination thereof. In one embodiment, the pH value of the culture environment for the first acclimation culture mentioned above may be about pH 5-8, such as about pH 5.5-7.5, about pH 6-7, about pH 5.0, about pH 5.5, about pH 6.0, about pH 6.5, about pH 7.0, about pH 7.5, about pH 8.0, but not limited thereto. In one specific embodiment, the pH value of the culture environment for the first acclimation culture mentioned above may be about pH 7.0.

Furthermore, in the first acclimation culture mentioned above, the composition and/or type of the culture medium for culturing the source microorganism mentioned above is/are not particularly limited, as long as the source microorganism mentioned above can grow therein. One skilled in the art can select a suitable medium based on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), etc., or any combination thereof.

In one embodiment, in the first acclimation culture mentioned above, the culture medium used for culturing the source microorganism mentioned above is a basal medium. The carbon source of the basal medium mentioned above may comprise, but is not limited to, glucose, glycerol, sucrose, fructose, etc., or any combination thereof. Moreover, in the basal medium mentioned above, the concentration of carbon source may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto. In one embodiment, the carbon source mentioned above may be glucose, and the concentration thereof may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto.

In one specific embodiment, in the first acclimation culture mentioned above, the culture medium used for culturing the source microorganism mentioned above is a basal medium, and the composition of the basal medium mentioned above may comprise glucose and phosphate buffered saline, but it is not limited thereto. Moreover, in this specific example, the concentration of glucose may be about 5 g/L.

In one embodiment, in the first acclimation culture mentioned above, the obtained at least one first acclimatized microorganism mentioned above is the at least one acclimatized microorganism mentioned above.

Moreover, in another embodiment, in the method for preparing a p-aminobenzoic acid-producing microorganism of the present disclosure mentioned above, the at least one acclimation culture may further comprise a second acclimation culture after the first acclimation culture, but it is not limited thereto.

In the second acclimation culture mentioned above, the at least one first acclimatized microorganism obtained from the first acclimation culture may be cultured in the presence of the at least one sulfonamide antibiotic mentioned above at a second concentration to obtain at least one second acclimatized microorganism.

Moreover, in the second acclimation culture mentioned above, the at least one sulfonamide antibiotic mentioned above may comprise a second sulfonamide antibiotic, but it is not limited thereto. The second sulfonamide antibiotic mentioned above may comprise, but is not limited to, sulfanilamide, sulfacetamide, sulfadiazine, sulfasalazine, sulfadimidine, sulfafurazole, etc., or any combination thereof. In one embodiment, the second sulfonamide antibiotic mentioned above may be sulfanilamide.

Furthermore, in one embodiment, the first sulfonamide antibiotic and the second sulfonamide antibiotic may be the same. In one specific embodiment, both of the foregoing first sulfonamide antibiotic and the foregoing second sulfonamide antibiotic are sulfanilamide.

In another embodiment, the foregoing first sulfonamide antibiotic and the foregoing second sulfonamide antibiotic may be different.

In the second acclimation culture mentioned above, the second concentration of the at least one sulfonamide antibiotic mentioned above may be about 250-5000 mg/L, about 500-4500 mg/L, about 750-4000 mg/L, about 800-3500 mg/L, about 1000-3000 mg/L, 250 mg/L, about 300 mg/L, about 400 mg/L, about 500 mg/L, about 600 mg/L, about 700 mg/L, about 800 mg/L, about 900 mg/L, about 1000 mg/L, about 1200 mg/L, about 1250 mg/L, about 1500 mg/L, about 2000 mg/L, about 2250 mg/L, about 2500 mg/L, about 3000 mg/L, about 3500 mg/L, about 4000 mg/L, about 4500 mg/L, about 5000 mg/L, but it is not limited thereto.

In one embodiment, the foregoing second concentration of the at least one sulfonamide antibiotic in the second acclimation culture mentioned above may be higher than the foregoing first concentration of the at least one sulfonamide antibiotic in the first acclimation culture mentioned above. In one specific embodiment, the foregoing second concentration of the at least one sulfonamide antibiotic in the second acclimation culture mentioned above is higher than the foregoing first concentration of the at least one sulfonamide antibiotic in the first acclimation culture mentioned above, and the foregoing first concentration of the at least one sulfonamide antibiotic in the first acclimation culture mentioned above may be about 50-1000 mg/L while the foregoing second concentration of the at least one sulfonamide antibiotic in the second acclimation culture mentioned above may be about 1000-3000 mg/L.

Moreover, the culturing time for the second acclimation culture is not particularly limited, depending on the type of microorganism cultured, the culture method (such as batch culture and continuous culture), external environment (such as external environment temperature, external environment humidity and culture container/tank size), culture conditions (such as culture temperature, culture volume, composition of culture medium, type of culture medium, pH value of the culture environment, stirring rate and aeration rate), microbial growth circumstances, etc., or any combination thereof. In one embodiment, the culturing time for the second acclimation culture mentioned above may be about 6-72 hours, such as about 12-66 hours, about 18-60 hours, about 24-54 hours, about 30-48 hours, about 12-48 hours, about 6 hours, about 12 hours, about 18 hours, about 24 hours, about 30 hours, about 48 hours, about 54 hours, about 60 hours, about 66 hours and about 72 hours, but it is not limited thereto. In one specific embodiment, the culturing time for the second acclimation culture mentioned above may be about 24 hours.

Similarly, the culture temperature for the second acclimation culture mentioned above is not particularly limited and may depend on the type of microorganism cultured, the culture method (such as batch culture and continuous culture), external environment (such as external environment temperature, external environment humidity and culture container/tank size), culture conditions (such as culture time, culture volume, composition of culture medium, type of culture medium, pH value of the culture environment, stirring rate and aeration rate), microbial growth circumstances, etc., or any combination thereof. In one embodiment, the culture temperature for the second acclimation culture mentioned above may be about 20-45° C., such as about 22-44° C., about 25-43° C., about 27-44° C., about 30-40° C., about 32-38° C., about 35-37° C., about 22-35° C., about 24-32° C., about 25-30° C., about 25° C., about 27° C., about 28° C., about 29° C., about 30° C., about 32° C., about 33° C., about 34° C., about 35° C., about 36° C., about 37° C., about 37.5° C., about 38° C., about 39° C., about 40° C., about 42° C. and about 45° C., but it is not limited thereto. In one specific embodiment, the culture temperature for the second acclimation culture mentioned above may be about 37° C.

The pH value of the culture environment for the second acclimation culture mentioned above is not particularly limited, and may depend on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), microbial growth circumstances, etc., or any combination thereof. In one embodiment, the pH value of the culture environment for the second acclimation culture mentioned above may be about pH 5-8, such as about pH 5.5-7.5, about pH 6-7, about pH 5.0, about pH 5.5, about pH 6.0, about pH 6.5, about pH 7.0, about pH 7.5, about pH 8.0, but not limited thereto. In one specific embodiment, the pH value of the culture environment for the second acclimation culture mentioned above may be about pH 7.0.

In the second acclimation culture mentioned above, the composition and/or type of the culture medium for culturing the at least one first acclimatized microorganism mentioned above obtained in the first acclimation culture mentioned above can grow therein. One skilled in the art can select a suitable medium based on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), etc., or any combination thereof.

In one embodiment, in the second acclimation culture mentioned above, the culture medium used for culturing the at least one first acclimatized microorganism mentioned above obtained in the first acclimation culture mentioned above is a basal medium. The carbon source of the basal medium mentioned above may comprise, but is not limited to, glucose, glycerol, sucrose, fructose, etc., or any combination thereof. Moreover, in the basal medium mentioned above, the concentration of carbon source may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto. In one embodiment, the carbon source mentioned above may be glucose, and the concentration thereof may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto.

In one specific embodiment, in the second acclimation culture mentioned above, the culture medium used for culturing the at least one first acclimatized microorganism mentioned above obtained in the first acclimation culture mentioned above is a basal medium, and the composition of the basal medium mentioned above may comprise glucose and phosphate buffered saline, but it is not limited thereto. Moreover, in this specific example, the concentration of glucose may be about 5 g/L.

In one embodiment, in the second acclimation culture mentioned above, the obtained at least one second acclimatized microorganism mentioned above is the at least one acclimatized microorganism mentioned above.

In addition, in another embodiment, the method for preparing a p-aminobenzoic acid-producing microorganism of the present disclosure mentioned above may further comprise a preliminary screening culture before the acclimation process is performed on the source microorganism with at least one sulfonamide antibiotic, but it is not limited thereto.

In the above-mentioned preliminary screening culture, the source microorganism mentioned above may be cultured in the presence of at least one sulfonamide antibiotic at a third concentration to obtain at least one screened source microorganism. In the above-mentioned preliminary screening culture, the obtained at least one screened source microorganism is used in the first acclimation culture.

In the preliminary screening culture mentioned above, the at least one sulfonamide antibiotic mentioned above may comprise a third sulfonamide antibiotic, but it is not limited thereto. The third sulfonamide antibiotic mentioned above may comprise, but is not limited to, sulfanilamide, sulfacetamide, sulfadiazine, sulfasalazine, sulfadimidine, sulfafurazole, etc., or any combination thereof. In one embodiment, the third sulfonamide antibiotic mentioned above may be sulfanilamide.

Furthermore, in one embodiment, the third sulfonamide antibiotic mentioned above, the first sulfonamide antibiotic mentioned above, and the second sulfonamide antibiotic mentioned above may be the same. In one specific embodiment, the third sulfonamide antibiotic mentioned above, the first sulfonamide antibiotic mentioned above, and the second sulfonamide antibiotic mentioned above all are sulfanilamide.

In another embodiment, the third sulfonamide antibiotic mentioned above, the first sulfonamide antibiotic mentioned above, and the second sulfonamide antibiotic mentioned above may be different from each other.

In the preliminary screening culture mentioned above, the third concentration of the at least one sulfonamide antibiotic mentioned above may be about 0.1-50 mg/L, about 0.5-40 mg/L, about 1-30 mg/L, about 2-25 mg/L, about 2.5-20 mg/L, 3-15 mg/L, about 5-10 mg/L, about 0.1 mg/L, about 0.2 mg/L, about 0.25 mg/L, about 0.5 mg/L, about 1 mg/L, about 1.5 mg/L, about 2 mg/L, about 2.5 mg/L, about 3 mg/L, about 4 mg/L, about 5 mg/L, about 8 mg/L, about 10 mg/L, about 15 mg/L, about 20 mg/L, about 25 mg/L, about 30 mg/L, about 40 mg/L, about 50 mg/L, etc., but it is not limited thereto.

In one embodiment, the foregoing third concentration of the at least one sulfonamide antibiotic in preliminary screening culture mentioned above may be lower than the foregoing first concentration of the at least one sulfonamide antibiotic in the first acclimation culture mentioned above and may be lower than the foregoing second concentration of the at least one sulfonamide antibiotic in the second acclimation culture mentioned above. In one specific embodiment, the foregoing third concentration of the at least one sulfonamide antibiotic in preliminary screening culture mentioned above may be lower than the foregoing first concentration of the at least one sulfonamide antibiotic in the first acclimation culture mentioned above and may be lower than the foregoing second concentration of the at least one sulfonamide antibiotic in the second acclimation culture mentioned above while the foregoing third concentration of the at least one sulfonamide antibiotic in preliminary screening culture mentioned above may be about 0.1-50 mg/L, the foregoing first concentration of the at least one sulfonamide antibiotic in the first acclimation culture mentioned above may be about 50-1000 mg/L and the foregoing second concentration of the at least one sulfonamide antibiotic in the second acclimation culture mentioned above may be about 1000-3000 mg/L.

Furthermore, the culturing time for the preliminary screening culture is not particularly limited, depending on the type of microorganism cultured, the culture method (such as batch culture and continuous culture), external environment (such as external environment temperature, external environment humidity and culture container/tank size), culture conditions (such as culture temperature, culture volume, composition of culture medium, type of culture medium, pH value of the culture environment, stirring rate and aeration rate), microbial growth circumstances, etc., or any combination thereof. In one embodiment, the culturing time for the preliminary screening culture mentioned above may be about 6-72 hours, such as about 12-66 hours, about 18-60 hours, about 24-54 hours, about 30-48 hours, about 12-48 hours, about 6 hours, about 12 hours, about 18 hours, about 24 hours, about 30 hours, about 48 hours, about 54 hours, about 60 hours, about 66 hours and about 72 hours, but it is not limited thereto. In one specific embodiment, the culturing time for the preliminary screening culture mentioned above may be about 24 hours.

Similarly, the culture temperature for the preliminary screening culture mentioned above is not particularly limited and may depend on the type of microorganism cultured, the culture method (such as batch culture and continuous culture), external environment (such as external environment temperature, external environment humidity and culture container/tank size), culture conditions (such as culture time, culture volume, composition of culture medium, type of culture medium, pH value of the culture environment, stirring rate and aeration rate), microbial growth circumstances, etc., or any combination thereof. In one embodiment, the culture temperature for the preliminary screening culture mentioned above may be about 20-45° C., such as about 22-44° C., about 25-43° C., about 27-44° C., about 30-40° C., about 32-38° C., about 35-37° C., about 22-35° C., about 24-32° C., about 25-30° C., about 25° C., about 27° C., about 28° C., about 29° C., about 30° C., about 32° C., about 33° C., about 34° C., about 35° C., about 36° C., about 37° C., about 37.5° C., about 38° C., about 39° C., about 40° C., about 42° C. and about 45° C., but it is not limited thereto. In one specific embodiment, the culture temperature for the preliminary screening culture mentioned above may be about 37° C.

The pH value of the culture environment for the preliminary screening culture mentioned above is not particularly limited, and may depend on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), microbial growth circumstances, etc., or any combination thereof. In one embodiment, the pH value of the culture environment for the preliminary screening culture mentioned above may be about pH 5-8, such as about pH 5.5-7.5, about pH 6-7, about pH 5.0, about pH 5.5, about pH 6.0, about pH 6.5, about pH 7.0, about pH 7.5, about pH 8.0, but not limited thereto. In one specific embodiment, the pH value of the culture environment for the preliminary screening culture mentioned above may be about pH 7.0.

In the preliminary screening culture mentioned above, the composition and/or type of the culture medium for culturing the source microorganism mentioned above is/are not particularly limited, as long as the source microorganism mentioned above can grow therein. One skilled in the art can select a suitable medium based on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), etc., or any combination thereof.

In one embodiment, in the preliminary screening culture mentioned above, the culture medium used for culturing the source microorganism mentioned above is a basal medium. The carbon source of the basal medium mentioned above may comprise, but is not limited to, glucose, glycerol, sucrose, fructose, etc., or any combination thereof. Moreover, in the basal medium mentioned above, the concentration of carbon source may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto. In one embodiment, the carbon source mentioned above may be glucose, and the concentration thereof may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L and about 30 g/L, but it is not limited thereto.

In one specific embodiment, in the preliminary screening culture mentioned above, the culture medium used for culturing the source microorganism mentioned above is a basal medium, and the composition of the basal medium mentioned above may comprise glucose and phosphate buffered saline, but it is not limited thereto. Furthermore, in this specific example, the concentration of glucose may be about 5 g/L.

In one embodiment, compared to the source microorganism mentioned above, the at least one p-aminobenzoic acid-producing microorganism obtained in the method for preparing a p-aminobenzoic acid-producing microorganism of the present disclosure may have, but is not limited to, overexpression of a gene related to citrate and malate biosynthesis, suppressive expression of a gene related to glutamine metabolism, etc., or any combination thereof.

The foregoing gene related to citrate and malate biosynthesis may include, but is not limited to, at least one of acnA, icd, glcB, etc. In one embodiment, the gene related to citrate and malate biosynthesis may include acnA, icd and glcB.

The above-mentioned gene related to the glutamine metabolism may include at least one of glsA, purF, pyrB, etc., but it is not limited thereto. In one embodiment, the above-mentioned gene related to glutamine metabolism may include glsA, purF and pyrB.

In one specific embodiment, compared to the above-mentioned source microorganism, the at least one p-aminobenzoic acid-producing microorganism obtained in the method for preparing a p-aminobenzoic acid-producing microorganism of the present disclosure may have, but is not limited to, overexpression of a gene related to citrate and malate biosynthesis and suppressive expression of a gene related to glutamine metabolism, wherein the gene related to citrate and malate biosynthesis may include acnA, icd and glcB, and the gene related to glutamine metabolism may include glsA, purF and pyrB. In this specific embodiment, the source microorganism in the method for preparing a p-aminobenzoic acid-producing microorganism of the present disclosure may be a genetically modified Escherichia coli BW25113. Also, in this specific embodiment, the foregoing genetically modified Escherichia coli BW25113 may comprise at least one exogenous gene, and the at least one exogenous gene may comprise pabA, pabB and pabC.

Furthermore, in another specific embodiment, the at least one p-aminobenzoic acid-producing microorganism obtained in the method for preparing a p-aminobenzoic acid-producing microorganism of the present disclosure may include, but is not limited to, Escherichia coli BW25113 JY33P5B, which was deposited at Bioresource Collection and Research Centre (BCRC) of Food Industry Research and Development Institute (FIRDI) (ROC.) on Aug. 17, 2022, with the deposit number BCRC 940697, and also was deposited at Leibniz Institute DSMZ-German Collection of Microorganisms and Cell Cultures on Aug. 23, 2022, with the deposit number DSM 34363.

Based on the foregoing, the present disclosure may also provide a p-aminobenzoic acid-producing microorganism prepared by any of the above-mentioned method for preparing a p-aminobenzoic acid-producing microorganism of the present disclosure.

In addition, the present disclosure can also provide another p-aminobenzoic acid-producing microorganism. The p-aminobenzoic acid-producing microorganism described herein is derived from a source microorganism. Compared to the foregoing source microorganism, the p-aminobenzoic acid-producing microorganism described herein may have the overexpression of a gene related to citrate and malate biosynthesis, suppressive expression of a gene related to glutamine metabolism, etc., or any combination thereof. Moreover, the p-aminobenzoic acid-producing microorganism described herein may have higher p-aminobenzoic acid titer compared to the above-mentioned source microorganism.

The aforementioned gene related to citrate and malate biosynthesis may include, but is not limited to, at least one of acnA, icd, glcB, etc. In one embodiment, the gene related to citrate and malate biosynthesis may include acnA, icd and glcB.

Furthermore, the above-mentioned gene related to glutamine metabolism may include at least one of glsA, purF, pyrB, etc., but it is not limited thereto. In one embodiment, the above-mentioned gene related to glutamine metabolism may include glsA, purF and pyrB.

In one embodiment, compared to the above-mentioned source microorganism, the above-mentioned p-aminobenzoic acid-producing microorganism of the present disclosure may have overexpression of a gene related to citrate and malate biosynthesis and suppressive expression of a gene related to glutamine metabolism, but it not limited thereto, wherein the above-mentioned gene related to citrate and malate biosynthesis may include acnA, icd and glcB, and the above-mentioned gene related to glutamine metabolism may include glsA, purF and pyrB.

The above-mentioned source microorganisms of the above-mentioned p-aminobenzoic acid-producing microorganism may be a microorganism without any genetic modification, or may also be a genetically modified microorganism, as long as the general biosynthetic pathway of folate in microorganisms mentioned above exists in the microorganism. In one embodiment, the source microorganism mentioned above may be a genetically modified microorganism.

The genetically modified microorganism mentioned above may contain at least one exogenous gene, but it is not limited thereto. The at least one exogenous gene contained in the genetically modified microorganism mentioned above may comprise at least one of pabA, pabB, pabC, etc., but is not limited thereto. In one embodiment, the at least one exogenous gene contained in the genetically modified microorganism mentioned above may comprise, but is not limited to, pabA, pabB and pabC.

There is no particular limitation on the form in which the at least one exogenous gene contained in the foregoing genetically modified microorganism is preset in the foregoing genetically modified microorganism, as long as the at least one of the exogenous genes mentioned above can be expressed in this genetically modified microorganism and does not negatively affect other endogenous genes. In one embodiment, the at least one exogenous gene mentioned above may be present in at least one vector contained in the genetically modified microorganism mentioned above, and example of the vector mentioned above may comprise, but is not limited to, a plasmid or a viral vector. In another embodiment, the at least one exogenous gene mentioned above may be inserted into the chromosomal DNA of the genetically modified microorganism mentioned above.

Moreover, for the type and example of the above-mentioned source microorganism of the above-mentioned p-aminobenzoic acid-producing microorganism of the present disclosure, the previous paragraphs which recite the relevant descriptions for the source microorganism for the method for preparing a p-aminobenzoic acid-producing microorganism of the present disclosure mentioned above can be referred, and therefore will not be repeated here.

In one embodiment, the foregoing source microorganism of the foregoing p-aminobenzoic acid-producing microorganism of the present disclosure may be Escherichia coli.

In another embodiment, the foregoing source microorganism of the foregoing p-aminobenzoic acid-producing microorganism of the present disclosure may be a genetically modified Escherichia coli. In this embodiment, the genetically modified Escherichia coli may contain at least one exogenous gene, but is not limited thereto. The at least one exogenous gene contained in the genetically modified Escherichia coli mentioned above may comprise, but is not limited to, least one of pabA, pabB and pabC, etc. In one specific embodiment, the at least one exogenous gene contained in the genetically modified Escherichia coli mentioned above may comprise pabA, pabB and pabC. The aforementioned genetically modified Escherichia coli may be genetically modified Escherichia coli BW25113. Also, in this specific embodiment, compared to the source microorganism, i.e., the genetically modified Escherichia coli mentioned above, the p-aminobenzoic acid-producing microorganism described herein may have overexpression of a gene related to citrate and malate biosynthesis and suppressive expression of a gene related to glutamine metabolism, wherein the foregoing gene related to citrate and malate biosynthesis may include acnA, icd and glcB while the foregoing gene related to glutamine metabolism may include glsA, purF and pyrB.

In another embodiment, the p-aminobenzoic acid-producing microorganism of the present disclosure mentioned above may include, but not limited to, Escherichia coli BW25113 JY33P5B, which was deposited at Bioresource Collection and Research Centre (BCRC) of Food Industry Research and Development Institute (FIRDI) (ROC.) on Aug. 17, 2022, with the deposit number BCRC 940697, and also was deposited at Leibniz Institute DSMZ-German Collection of Microorganisms and Cell Cultures on Aug. 23, 2022, with the deposit number DSM 34363.

In addition, based on the foregoing, the present disclosure may also provide a Escherichia coli that produces p-aminobenzoic acid, that is, Escherichia coli BW25113 JY33P5B, which was deposited at Bioresource Collection and Research Centre (BCRC) of Food Industry Research and Development Institute (FIRDI) (ROC.) on Aug. 17, 2022, with the deposit number BCRC 940697, and also was deposited at Leibniz Institute DSMZ-German Collection of Microorganisms and Cell Cultures on Aug. 23, 2022, with the deposit number DSM 34363.

Furthermore, according to the foregoing, the present invention can further provide a method for producing p-aminobenzoic acid. The method for producing p-aminobenzoic acid of the present disclosure mentioned above may include, but is not limited to, a microorganism culturing step.

In the foregoing microorganism culturing step, a p-aminobenzoic acid-producing microorganism can be cultured so that the above-mentioned p-aminobenzoic acid-producing microorganism can produce p-aminobenzoic acid, wherein the above-mentioned p-aminobenzoic acid-producing microorganism may include, but is not limited to, any of the p-aminobenzoic acid-producing microorganisms of the present disclosure mentioned above, any of the p-aminobenzoic acid-producing Escherichia coli of the present disclosure mentioned above, etc., or any combination thereof. In one specific embodiment, the above-mentioned p-aminobenzoic acid-producing microorganism is Escherichia coli BW25113 JY33P5B, which was deposited at Bioresource Collection and Research Centre (BCRC) of Food Industry Research and Development Institute (FIRDI) (ROC.) on Aug. 17, 2022, with the deposit number BCRC 940697, and also was deposited at Leibniz Institute DSMZ-German Collection of Microorganisms and Cell Cultures on Aug. 23, 2022, with the deposit number DSM 34363.

Also, in the above-mentioned microorganism culturing step, the above-mentioned p-aminobenzoic acid-producing microorganism may be cultured in a culture medium to obtain a cultured medium, and the foregoing cultured medium contains p-aminobenzoic acid. Namely, the above-mentioned p-aminobenzoic acid-producing microorganism can secrete the produced p-aminobenzoic acid out of cells.

The culture temperature used in the microorganism culturing step mentioned above is not particularly limited and may depend on the type of microorganism cultured, the culture method (such as batch culture and continuous culture), external environment (such as external environment temperature, external environment humidity and culture container/tank size), culture conditions (such as culture time, culture volume, composition of culture medium, type of culture medium, pH value of the culture environment, stirring rate and aeration rate), microbial growth circumstances, etc., or any combination thereof. In one embodiment, the culture temperature for the first acclimation culture mentioned above may be about 20-45° C., such as about 22-44° C., about 25-43° C., about 27-44° C., about 30-40° C., about 32-38° C., about 35-37° C., about 22-35° C., about 24-32° C., about 25-30° C., about 25° C., about 27° C., about 28° C., about 29° C., about 30° C., about 32° C., about 33° C., about 34° C., about 35° C., about 36° C., about 37° C., about 37.5° C., about 38° C., about 39° C., about 40° C., about 42° C. and about 45° C., but it is not limited thereto. In one specific embodiment, the culture temperature for the microorganism culturing step mentioned above may be about 27° C.

In addition, the culturing time used in the microorganism culturing step mentioned above is not particularly limited, depending on the type of microorganism cultured, the culture method (such as batch culture and continuous culture), external environment (such as external environment temperature, external environment humidity and culture container/tank size), culture conditions (such as culture temperature, culture volume, composition of culture medium, type of culture medium, pH value of the culture environment, stirring rate and aeration rate), microbial growth circumstances, etc., or any combination thereof. In one embodiment, the culturing time used in the microorganism culturing step mentioned above may be about 6-72 hours, such as about 12-66 hours, about 18-60 hours, about 24-54 hours, about 30-48 hours, about 12-48 hours, about 6 hours, about 12 hours, about 18 hours, about 24 hours, about 30 hours, about 48 hours, about 54 hours, about 60 hours, about 66 hours and about 72 hours, but it is not limited thereto. In one specific embodiment, the culturing time used in the microorganism culturing step mentioned above may be about 24 hours. In another specific embodiment, the culturing time used in the microorganism culturing step mentioned above may be about 48 hours.

In the microorganism culturing step mentioned above, the pH value of the culture environment is not particularly limited, and may depend on the type of microorganism cultured, the culture method (such as batch culture and continuous culture), external environment (such as external environment temperature, external environment humidity and culture flask/fermentor size), culture conditions (such as culture time, culture temperature, culture volume, composition of culture medium, type of culture medium, stirring rate and aeration rate), microbial growth circumstances, etc., or any combination thereof. In one embodiment, in the microorganism culturing step mentioned above, the pH value of the culture environment may be about pH 5-8, such as about pH 5.5-7.5, about pH 6-7, about pH 5.0, about pH 5.5, about pH 6.0, about pH 6.5, about pH 7.0, about pH 7.5, about pH 8.0, but not limited thereto. In one specific embodiment, in the microorganism culturing step mentioned above, the pH value of the culture environment may be about pH 7.0.

Furthermore, in the microorganism culturing step mentioned above, the composition and/or type of the culture medium for culturing the above-mentioned p-aminobenzoic acid-producing microorganism is/are not particularly limited, as long as the above-mentioned p-aminobenzoic acid-producing microorganism can grow therein. One skilled in the art can select a suitable medium based on the type of microorganism cultured, the culture method (such as batch culture and continuous culture), external environment (such as external environment temperature, external environment humidity and culture container/tank size), culture conditions (such as culture time, culture temperature, culture volume, composition of culture medium, type of culture medium, stirring rate and aeration rate), etc., or any combination thereof.

In one embodiment, in the microorganism culturing step mentioned above, the culture medium used for culturing the above-mentioned p-aminobenzoic acid-producing microorganism is a basal medium. The carbon source of the basal medium mentioned above may comprise, but is not limited to, glucose, glycerol, sucrose, fructose, etc., or any combination thereof. Moreover, in the basal medium mentioned above, the concentration of carbon source may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L, about 30 g/L, but it is not limited thereto. In one embodiment, the carbon source mentioned above may be glucose, and the concentration thereof may be about 0.5-30 g/L, such as about 1-30 g/L, about 5-25 g/L, about 10-20 g/L, about 2-8 g/L, about 3-7 g/L, about 4-6 g/L, about 1 g/L, about 2 g/L, about 2.5 g/L, about 3 g/L, about 4 g/L, about 5 g/L, about 6 g/L, about 7 g/L, about 8 g/L, about 9 g/L, about 10 g/L, about 12.5 g/L, about 15 g/L, about 20 g/L, about 25 g/L, about 25.5 g/L, about 27.5 g/L, about 30 g/L, but it is not limited thereto.

In one specific embodiment, in the microorganism culturing step mentioned above, the culture medium used for culturing the above-mentioned p-aminobenzoic acid-producing microorganism is a basal medium, and the composition of the basal medium mentioned above may comprise glucose and phosphate buffered saline, but it is not limited thereto. Moreover, in this specific example, the concentration of glucose may be about 5 g/L.

In one embodiment, the method for producing p-aminobenzoic acid of the present disclosure may further include an isolation step after the microorganism culturing step, but is not limited thereto.

In the foregoing isolation step, p-aminobenzoic acid may be isolated from the cultured medium mentioned above. The method of isolating p-aminobenzoic acid from the cultured medium mentioned above is not particularly limited, as long as isolated p-aminobenzoic acid can be obtained. The method for isolating p-aminobenzoic acid from the cultured medium mentioned above may include, but is not limited to, high-performance liquid chromatography (HPLC).

### EXAMPLES

### Example 1: Genetically Modified Bacterial Strains

1. Plasmid Construction

According to the schematic diagram of the general biosynthetic pathway of folate in microorganisms shown in FIG. 1, the genes pabA, pabB and pabC are all genes of enzymes related to biosynthesis of p-aminobenzoic acid (PABA) in microorganisms.

Nucleotide sequence of gene pabA (NCBI Gene ID: 947873) (SEQ ID NO. 1, the amino acid sequence encoded thereby is SEQ ID NO. 2), nucleotide sequence of gene pabB (NCBI Gene ID: 946337) (SEQ ID NO: 3, the amino acid sequence encoded thereby is SEQ ID NO. 4), the nucleotide sequence of gene pabC (NCBI Gene ID: 946647) (SEQ ID NO. 5, the amino acid sequence encoded thereby is SEQ ID NO. 6) from the Escherichia coli genome were obtained from NCBI (National Center for Biotechnology Information).

The primer sequences of the gene pabA, the primer sequences of the gene pabB and the primer sequences of the gene pabC were respectively designed.

The information of each primer sequence is as follows:

(1) Gene pabA
Forward primer:
(SEQ ID NO. 7)
GGACGCACTGACCGAATTCATTAAAGAGGAGAAAGGTACCATGATCCTG
CTTATAGATAA
Reverse primer:
(SEQ ID NO. 8)
GGAATTCGATATCAAGCTTATCGATACCGTCGACCTCGAGTCAGCGATG
CAGGAAATTAG
(2) Gene pabB
Forward primer:
(SEQ ID NO. 9)
GGACGCACTGACCGAATTCATTAAAGAGGAGAAAGGTACCATGAAGACG
TTATCTCCCGC
Reverse primer:
(SEQ ID NO. 10)
GGAATTCGATATCAAGCTTATCGATACCGTCGACCTCGAGTTACTTCTC
CAGTTGCTTCA
(3) Gene pabC
Forward primer:
(SEQ ID NO. 11)
GGACGCACTGACCGAATTCATTAAAGAGGAGAAAGGTACCATGTTCTTA
ATTAACGGTCA
Reverse primer:
(SEQ ID NO. 12)
GGAATTCGATATCAAGCTTATCGATACCGTCGACCTCGAGCTAATTCGG
GCGCTCACAAA

The foregoing primer sequences that conform to the 5′ end and 3′ end of the gene insertion position in plasmid pZE21-MCS-1.

Genome DNA of Escherichia coli BW25113 was extracted, and the above-mentioned gene pabA, gene pabB, and gene pabC were respectively amplified by polymerase chain reactions with the primers designed mentioned above, and then purified by electrophoresis to obtain the amplified fragment of gene pabA, the amplified fragment of gene pabB and the amplified fragment of gene pabC, respectively.

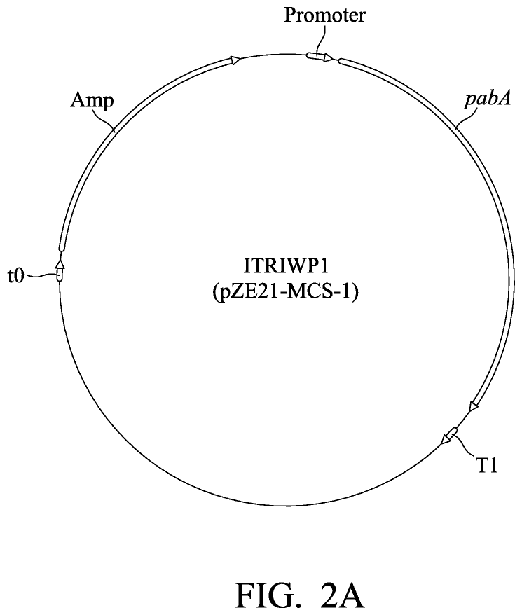

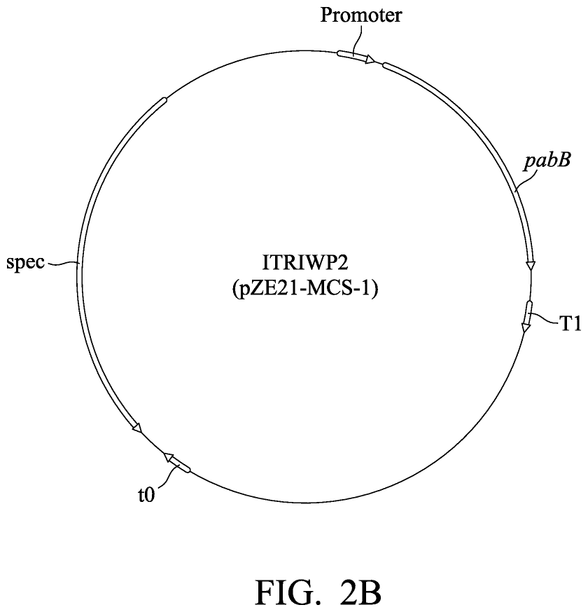

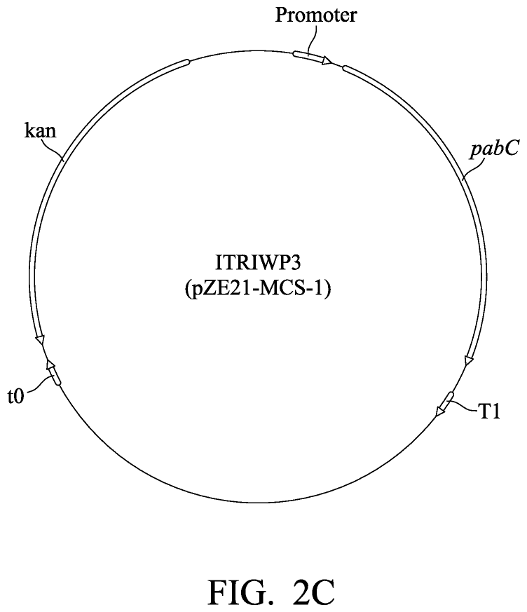

The obtained amplified fragment of gene pabA along with an ampicillin-resistant gene, the obtained amplified fragment of gene pabB along with a spectinomycin-resistant gene and the amplified fragment of the and the pabC gene along with a kamycin-resistant gene were respectively combined with the plasmids pZE21-MCS-1 to obtain the plasmid ITRIWP1 with the amplified fragment of gene pabA (containing the ampicillin-resistant gene), plasmid ITRIWP2 with the amplified fragment of gene pabB (containing the spectinomycin resistance gene), and plasmid ITRIWP3 with the amplified fragment of gene pabC (containing the kanamycin resistance gene). FIGS. 2A, 2B and 2C show the design of plasmid ITRIWP1, plasmid ITRIWP2 and plasmid ITRIWP3, respectively.

2. Genetic Modification of Bacterial Strain

Competent cells of Escherichia coli BW25113 were prepared. Next, plasmid ITRIWP1, plasmid ITRIWP2 and plasmid ITRIWP3 were transformed into the prepared Escherichia coli competent cells.

Then, the above-mentioned cells are cultured, and screened with antibiotics that meet the tolerance of the plasmids, to screen out bacterial strain containing the above-mentioned plasmid ITRIWP1, plasmid ITRIWP2 and plasmid ITRIWP3.

Finally, the screened bacterial strain is further confirmed whether it contains the gene fragments to be inserted through polymerase chain reactions. After confirmation, the obtained bacterial strain was the genetically modified Escherichia coli in this experiment, and named Escherichia coli BW.

### Example 2: Preparation of p-Aminobenzoic Acid-Producing Bacterial Strain

1. Material and Method

(1) Culture Medium

Formula A: LB culture medium (Luria broth, is the nutrient medium commonly used in microbial culture), ampicillin (100 mg/mL), spectinomycin (40 mg/mL) and kanamycin (50 mg/mL mL).

Formula B: glucose 5 g/L, phosphate buffer (PB), ampicillin (100 mg/mL), spectinomycin (40 mg/mL) and kanamycin (50 mg/mL).

(2) Color Development of p-Aminobenzoic Acid on Solid Medium

High rapid screening of p-aminobenzoic acid producing bacterial strains can be achieved by making p-aminobenzoic acid produced and secreted by the bacteria to directly develop color on the solid medium on which the bacteria grow, comparing the coloration of each colony, and selecting colonies with darker color. The steps of the above-mentioned color development method are as follows.

(i) Placing the culture plate on which bacterial colonies grow in a fume hood to dry for 30 minutes.

(ii) Adding 3 mL of 0.1% NaNO₂ to the culture plate and allowing to stand for 3 minutes.

(iii) Next, adding 3 mL of 0.5% ammonium sulfamate to the culture plate, and allowing to stand for 2 minutes.

(iv) Then, adding 1.5 mL of 0.1% hydrochloride of N-(naphyl)ethylenediamine (NED) to the culture plate and allowing to stand for 10 minutes.

(v) Afterwards, washing the culture plate with 5 mL of primary water.

(vi) Finally, adding 5 mL of 0.06N HCl to the culture plate and allowing to stand for 5 minutes to allow p-aminobenzoic acid to develop color. Colonies with darker red color are colonies with higher titer of p-aminobenzoic acid.

(3) Quantification of p-Aminobenzoic Acid

Quantification of p-aminobenzoic acid was performed by high-performance liquid chromatography (HPLC).

(i) Analytical Conditions of High-Performance Liquid Chromatography
Column: Japan GL SCIENCES Inertsustain C18 analysis column (5 m, 4.6×250 mm).
Mobile phase: 5 mM sodium 1-octanesulfonate, 10 mM KH₂PO₄ and 0.2% H₃PO₄ in H₂O/methanol (40/60).
Flow rate: 1 mL/minute.
Temperature: 26° C.
Detector: UV.
Detection wavelength: 203 nm.

(ii) Establishment of p-Aminobenzoic Acid Standard Curve

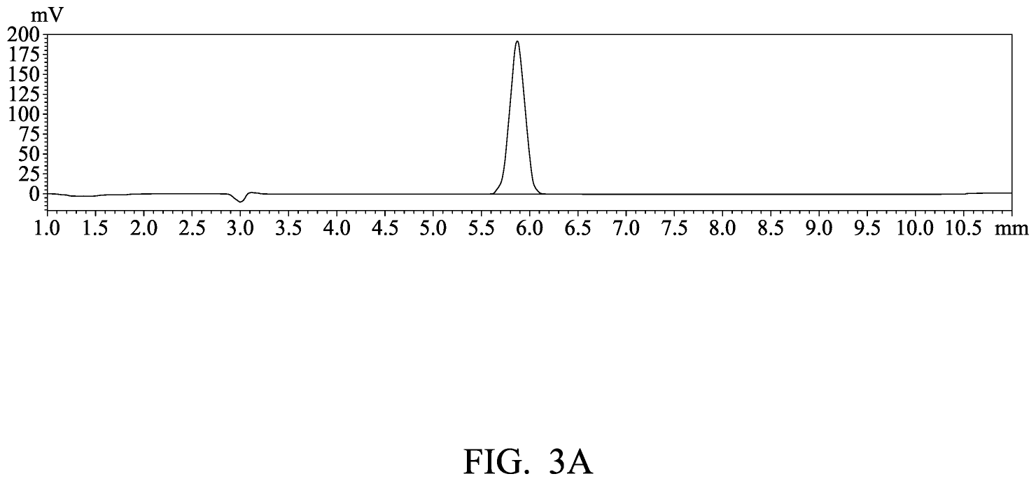

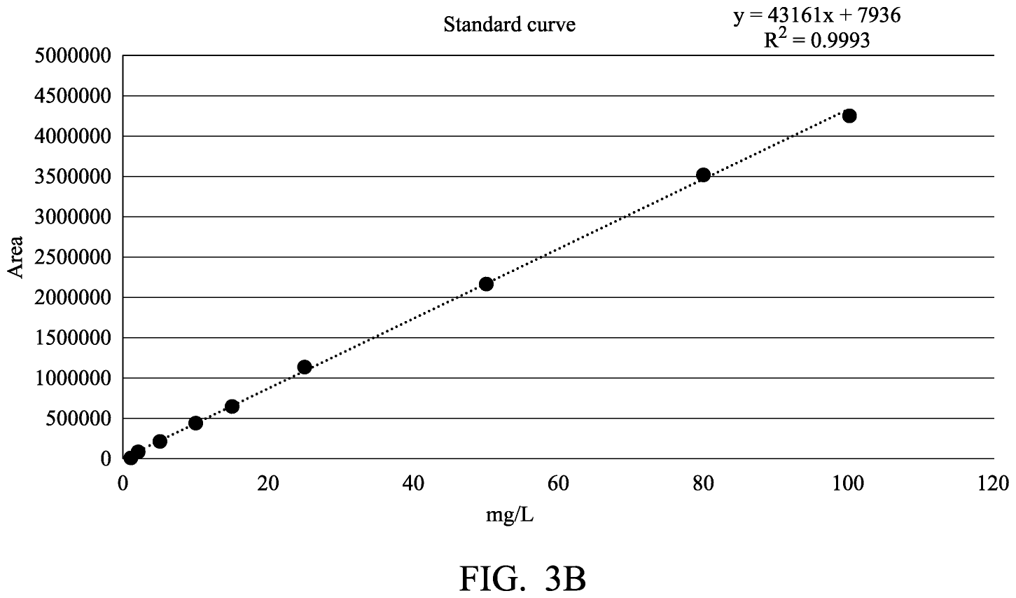

P-aminobenzoic acid was dissolved in water, and injected into the column of the above-mentioned high-performance liquid chromatography to perform high-performance liquid chromatography. The peak of p-aminobenzoic acid appeared at 5.9 minutes (FIG. 3A), showing that the standard curve of p-aminobenzoic acid can be established under this condition.

After that, standard curve (FIG. 3B) was established with p-aminobenzoic acid standard solution of different concentrations, and the range of standard curve is 1-100 mg/L.

2. Preliminary Screening of Bacterial Strain

Escherichia coli BW was picked out for a single colony with an inoculation loop, inoculated in the culture medium solution prepared by formula A and cultured at 37° C.

After culturing for 24 hours, the suspension of Escherichia coli BW was spread on a culture plate containing formula A and 1.5% agar, and cultured overnight at 37° C. Afterwards, 3 single colonies were randomly selected from the colonies grown on the culture plate, and named as Escherichia coli BW1, BW2 and BW3, respectively.

Escherichia coli BW1, BW2 and BW3 were respectively cultured in 14 mL culture tubes containing 2 mL of culture medium solution prepared by formula A and cultured overnight at 37° C.

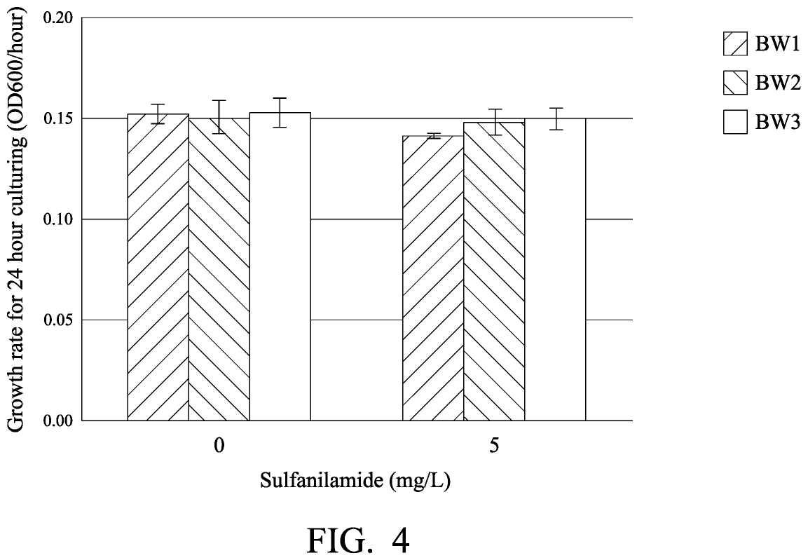

After that, Escherichia coli BW1, BW2 and BW3 suspensions obtained were inoculated in shake flasks containing formula B, 0 or 5 mg/L sulfanilamide and 0.5 mM isopropyl thiogalactoside, and cultured at pH 7.0, 37° C. for 24 hours, respectively. During the culturing period, the OD600 of the bacterial suspension was measured with a spectrophotometer to convert the bacterial content, and the bacterial growth rate within 24 hours was further calculated. The results are shown in Table 1 and FIG. 4.

In addition, after the foregoing 24 hours incubation, the bacterial suspensions in foregoing the shake flask that contained formula B, 5 mg/L sulfanilamide and 0.5 mM isopropyl thiogalactoside and was inoculated with Escherichia coli BW1, the bacterial suspensions in foregoing the shake flask that contained formula B, 5 mg/L sulfanilamide and 0.5 mM isopropyl thiogalactoside and was inoculated with Escherichia coli BW2, and the bacterial suspensions in foregoing the shake flask that contained formula B, 5 mg/L sulfanilamide and 0.5 mM isopropyl thiogalactoside and was inoculated with Escherichia coli BW3 were spread on culture plates containing formula B, 5 mg/L sulfanilamide, 0.5 mM isopropyl thiogalactoside and 1.5% agar to obtain Plate BW1, Plate BW2 and Plate BW3, respectively, and cultured overnight at 27° C. After that, color development of p-aminobenzoic acid on solid medium was performed on the above-mentioned Plate BW1, Plate BW2 and Plate BW3, and the colonies with the darkest color were selected on the Plate BW1, Plate BW2 and Plate BW3, respectively. The colonies selected from Plate BW1, Plate BW2 and Plate BW3 were named Escherichia coli JY1, JY2, and JY3, respectively.

TABLE 1
Growth rates of Escherichia coli BW1, BW2 and BW3

| | |
|---|---|
| | Growth rate (OD600/hour) |
| Bacterial | Sulfanilamide |

| strain | 0 mg/L | 5 mg/L |
|---|---|---|
| BW1 | 0.152 ± 0.005 | 0.141 ± 0.001 |
| BW2 | 0.150 ± 0.008 | 0.148 ± 0.005 |
| BW3 | 0.153 ± 0.007 | 0.150 ± 0.006 |

3. Screening by Low Concentration Sulfanilamide

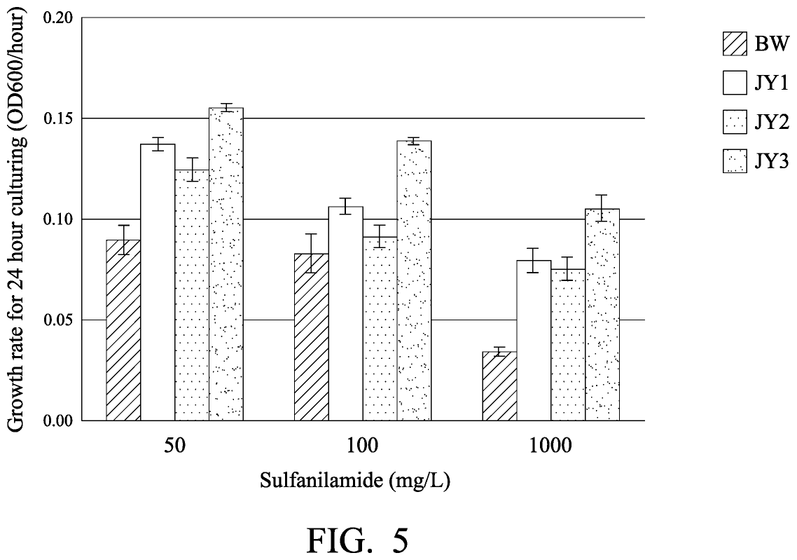

The suspensions of above-mentioned obtained Escherichia coli JY1, JY2 and JY3 and Escherichia coli BW were inoculated in shake flasks containing formula B, 50, 100 or 1000 mg/L sulfanilamide and 0.5 mM isopropyl thiogalactoside, and cultured at pH 7.0, 37° C. for 24 hours, respectively. During the culturing period, the OD600 of the bacterial suspension was measured with a spectrophotometer to convert the bacterial content, and the bacterial growth rate within 24 hours was further calculated. The results are shown in Table 2 and FIG. 5.

In addition, after the foregoing 24 hours culturing, the bacterial suspensions in foregoing the shake flask that contained formula B, 1000 mg/L sulfanilamide and 0.5 mM isopropyl thiogalactoside and was inoculated with Escherichia coli JY3 was spread on culture plates containing formula B, 1000 mg/L sulfanilamide, 0.5 mM isopropyl thiogalactoside and 1.5% agar to obtain Plate JY3 and cultured overnight at 27° C. After that, color development of p-aminobenzoic acid on solid medium was performed on the above-mentioned Plate JY3, and 3 colonies with the darkest color were selected on the Plate JY3. The 3 colonies selected from Plate JY3 were named Escherichia coli JY31, JY32, and JY33, respectively.

TABLE 2
Growth rates of Escherichia coli JY1, JY2 and JY3

| | |
|---|---|
| | Growth rate (OD600/hour) |
| Bacterial | Sulfanilamide |

| strain | 50 mg/L | 100 mg/L | 1000 mg/L |
|---|---|---|---|
| BW | 0.090 ± 0.007 | 0.083 ± 0.010 | 0.035 ± 0.002 |
| JY1 | 0.137 ± 0.003 | 0.107 ± 0.004 | 0.080 ± 0.006 |
| JY2 | 0.125 ± 0.006 | 0.092 ± 0.006 | 0.086 ± 0.006 |
| JY3 | 0.156 ± 0.002 | 0.139 ± 0.002 | 0.105 ± 0.007 |

4. Screening by High Concentration Sulfanilamide

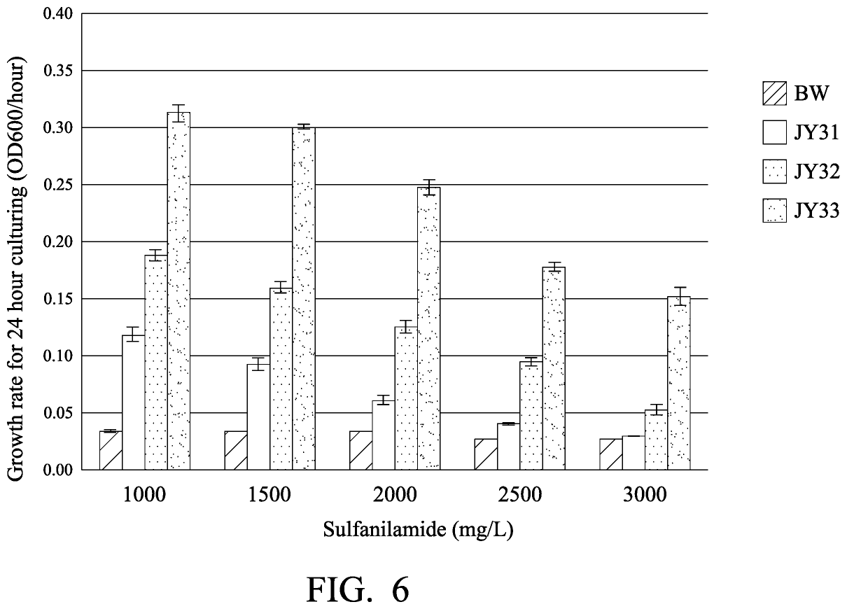

The suspensions of above-mentioned obtained Escherichia coli JY31, JY32 and JY33 and Escherichia coli BW were inoculated in shake flasks containing formula B, 1000, 1500, 2000, 2500 or 3000 mg/L sulfanilamide and 0.5 mM isopropyl thiogalactoside, and cultured at pH 7.0, 37° C. for 24 hours, respectively. During the culturing period, the OD600 of the bacterial suspension was measured with a spectrophotometer to convert the bacterial content, and the bacterial growth rate within 24 hours was further calculated. The results are shown in Table 3 and FIG. 6.

In addition, after the foregoing 24 hours culturing, the bacterial suspensions in foregoing the shake flask that contained formula B, 3000 mg/L sulfanilamide and 0.5 mM isopropyl thiogalactoside and was inoculated with Escherichia coli JY33 was spread on culture plates containing formula B, 3000 mg/L sulfanilamide, 0.5 mM isopropyl thiogalactoside and 1.5% agar to obtain Plate JY33 and cultured overnight at 27° C. After that, color development of p-aminobenzoic acid on solid medium was performed on the above-mentioned Plate JY33, and 5 colonies with the darkest color were selected on the Plate JY33. The 5 colonies selected from Plate JY33 were named Escherichia coli TT's Docket No.: 9044C-A28636US/Daphne/Dean/F JY33P1B, JY33P2B, JY33P3B, JY33P4B and JY33P5B, respectively.

TABLE 3
Growth rates of Escherichia coli JY31, JY32 and JY33.

| | Growth rate (OD600/hour) | | | | |
|---|---|---|---|---|---|
| Bacterial | Sulfanilamide | | | | |
| strain | 1000 mg/L | 1500 mg/L | 2000 mg/L | 2500 mg/L | 3000 mg/L |
| BW | 0.034 ± 0.001 | 0.033 ± 0.001 | 0.033 ± 0.000 | 0.027 ± 0.001 | 0.026 ± 0.001 |
| JY31 | 0.118 ± 0.007 | 0.092 ± 0.005 | 0.061 ± 0.005 | 0.040 ± 0.001 | 0.030 ± 0.001 |
| JY32 | 0.188 ± 0.005 | 0.160 ± 0.005 | 0.125 ± 0.006 | 0.095 ± 0.004 | 0.052 ± 0.005 |
| JY33 | 0.313 ± 0.008 | 0.300 ± 0.002 | 0.248 ± 0.007 | 0.178 ± 0.004 | 0.152 ± 0.008 |

5. Screening of Bacterial Strains with High Titer p-Aminobenzoic Acid

The suspensions of above-mentioned obtained Escherichia coli JY33P1B, JY33P2B, JY33P3B, JY33P4B and JY33P5B and Escherichia coli BW are respectively inoculated in the shake flask containing Formula B and 0.5 mM isopropyl thiogalactoside, and cultured at pH 7.0 and 27° C. for 24 or 48 hours.

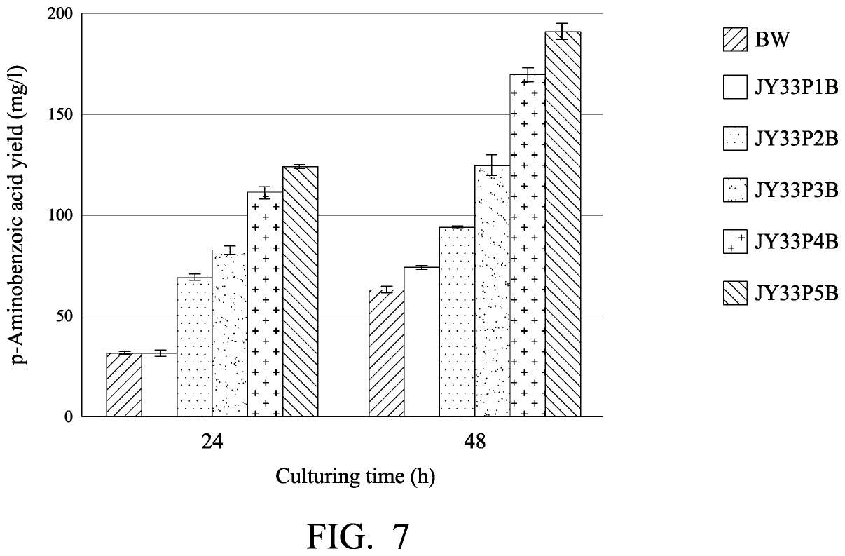

After the culturing was completed, the p-aminobenzoic acid content of the cultured medium solution for each bacterial strain was determined with high-performance liquid chromatography (HPLC). The results are shown in Table 4 and FIG. 7.

Table 4 and the FIG. 7 show that the production titer of p-aminobenzoic acid of Escherichia coli JY33P5B is 191.08 mg/L, and this is the highest p-aminobenzoic acid titer among the 5 bacterial strains. Compared to the original bacterial strain (Escherichia coli BW), the titer of p-aminobenzoic acid is increased by 3 times.

Escherichia coli JY33P5B was preserved with glycerol (glycerol: bacterial suspension=1:1), and was deposited at Bioresource Collection and Research Centre (BCRC) of Food Industry Research and Development Institute (FIRDI) (ROC.) on Aug. 17, 2022, with the deposit number BCRC 940697, and also deposited at Leibniz Institute DSMZ-German Collection of Microorganisms and Cell Cultures on Aug. 23, 2022, with the deposit number DSM 34363.

TABLE 4
Production titer of p-aminobenzoic acid in bacterial
strains with high sulfanilamide tolerance

| | |
|---|---|
| Bacterial | Production titer of p-aminobenzoic acid (mg/L) |

| strain | 24 hours | 48 hours |
|---|---|---|
| BW | 31.58 ± 0.47 | 63.10 ± 1.43 |
| JY33P1B | 31.35 ± 1.69 | 74.14 ± 1.03 |
| JY33P2B | 69.11 ± 1.40 | 93.82 ± 0.33 |
| JY33P3B | 82.61 ± 1.95 | 124.71 ± 5.26 |
| JY33P4B | 110.98 ± 2.93 | 169.34 ± 3.85 |
| JY33P5B | 123.80 ± 0.91 | 191.08 ± 3.81 |

### Example 3

Gene Expression Differences

Difference analysis of messenger RNA (mRNA) expression was performed on Escherichia coli JY33P5B and the original bacterial strain (Escherichia coli BW) through next-generation sequencing (NGS), and genes that are differentially expressed in the two bacterial strains and related to p-aminobenzoic acid biosynthesis were screened out. The results are shown in Table 5.

TABLE 5
Genes differentially expressed in Escherichia coli
JY33P5B and in the original strain (Escherichia coli
BW) and related to p-aminobenzoic acid biosynthesis

| | | | Fold, compared to the |
|---|---|---|---|
| Gene | Pathway | Expression | original strain |
| acnA | Citrate biosynthesis | Upregulation | 2.3 |
| icd | | | 2.4 |
| glcB | Malate biosynthesis | Upregulation | 5 |
| glsA | Glutamine | Downregulation | 12 |
| purF | metabolism | | 2.44 |
| pyrB | | | 2 |

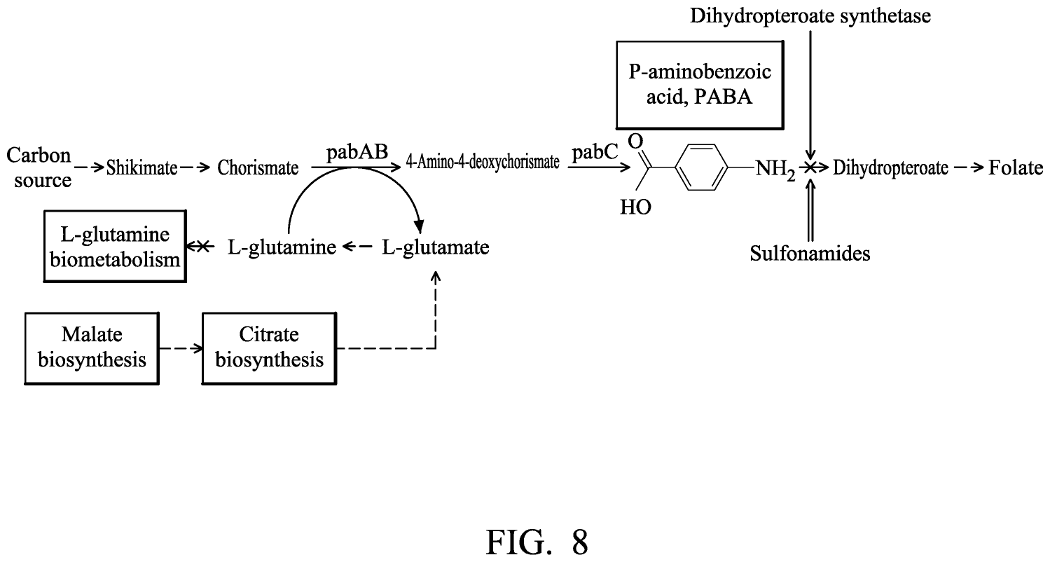

According to Table 5, compared to the original strain, the expressions of genes related to citrate and malate biosynthesis of Escherichia coli JY33P5B were increased, while the expressions of genes related to glutamine metabolism were suppressed. Combining the results mentioned above with the schematic diagram of the general biosynthetic pathway of folate in microorganisms shown in FIG. 1, the biosynthetic pathway of p-aminobenzoic acid in Escherichia coli JY33P5B can be inferred, as shown in FIG. 8.

Please refer to FIG. 8. According to FIG. 8, in Escherichia coli JY33P5B, in the biosynthetic pathway of p-aminobenzoic acid, chorismate and glutamic acid are used as substrates under the catalysis of the enzyme encoded by the gene pabAB to produce 4-amino-4-deoxychorismate required for synthesis of p-aminobenzoic acid.

Citrate and malate can be used for the synthesis of glutamate required for the synthesis of glutamine, and thus in Escherichia coli JY33P5B, increase of the expression of genes related to the citrate and malate biosynthesis can increase the citrate and malate biosynthesis while the metabolism pathway of glutamine is inhibited, allowing more glutamine to enter the biosynthesis pathway of p-aminobenzoic acid, thereby increasing the production of the p-aminobenzoic acid.

The experimental results mentioned above have proved that the method for preparing a p-aminobenzoic acid-producing microorganism of the present disclosure can indeed drive the evolution of the metabolic pathways of microorganisms and/or affect the expression of specific genes of microorganisms, thereby obtaining high-titer p-aminobenzoic acid-producing microorganisms.

It will be apparent to those skilled in the art that various modifications and variations can be made to the disclosed embodiments. It is intended that the specification and examples be considered as exemplary only, with a true scope of the disclosure being indicated by the following claims and their equivalents.

What is claimed is:

1. A p-aminobenzoic acid (PABA)-producing microorganism, obtained by a method for preparing a p-aminobenzoic acid-producing microorganism,
wherein the method for preparing a p-aminobenzoic acid-producing microorganism comprises:
(a) performing an acclimation process on a source microorganism with at least one sulfonamide antibiotic to obtain at least one acclimatized microorganism; and
(b) screening out at least one p-aminobenzoic acid-producing microorganism from the at least one acclimatized microorganism,
wherein the at least one p-aminobenzoic acid-producing microorganism has a higher p-aminobenzoic acid titer than the source microorganism.

2. The p-aminobenzoic acid-producing microorganism as claimed in claim 1, wherein a concentration of the at least one sulfonamide antibiotic is 10-5000 mg/L.

3. The p-aminobenzoic acid-producing microorganism as claimed in claim 1, wherein the at least one sulfonamide antibiotic comprises at least one of the following antibiotics:
sulfanilamide, sulfacetamide, sulfadiazine, sulfasalazine, sulfadimidine and sulfafurazole.

4. The p-aminobenzoic acid-producing microorganism as claimed in claim 1, wherein the at least one sulfonamide antibiotic is sulfanilamide.

5. The p-aminobenzoic acid-producing microorganism as claimed in claim 1, wherein the source microorganism comprises Escherichia coli, Corynebacterium glutamicum, Bacillus subtilis, Pseudomonas putida, Yarrowia lipolytica, Saccharomyces cerevisiae or Pichia pastoris.

6. The p-aminobenzoic acid-producing microorganism as claimed in claim 1, wherein the source microorganism is a genetically modified Escherichia coli, and the genetically modified Escherichia coli comprises at least one exogenous gene, and the at least one exogenous gene comprises pabA, pabB and pabC.

7. The p-aminobenzoic acid-producing microorganism as claimed in claim 1, wherein the acclimation process comprises:
at least one acclimation culture, and the at least one acclimation culture comprises:
a first acclimation culture;
wherein in the first acclimation culture, in the presence of the at least one sulfonamide antibiotic at a first concentration, culturing the source microorganism to obtain at least one first acclimatized microorganism, and
wherein the at least one sulfonamide antibiotic comprises a first sulfonamide antibiotic, and the first concentration is 10-3000 g/L.

8. The p-aminobenzoic acid-producing microorganism as claimed in claim 7, wherein the first sulfonamide antibiotic comprises at least one of the following antibiotics:
sulfanilamide, sulfacetamide, sulfadiazine, sulfasalazine, sulfadimidine and sulfafurazole.

9. The p-aminobenzoic acid-producing microorganism as claimed in claim 7, wherein the at least one first acclimatized microorganism is the at least one acclimatized microorganism.

10. The p-aminobenzoic acid-producing microorganism as claimed in claim 7, wherein the at least one acclimation culture further comprises:
a second acclimation culture which is performed after the first acclimation culture,
wherein in the second acclimation culture, in the presence of the at least one sulfonamide antibiotic at a second concentration, culturing the at least one first acclimatized microorganism to obtain at least one second acclimatized microorganism, and
wherein the at least one sulfonamide antibiotic comprises a second sulfonamide antibiotic, and the second concentration is 250-5000 g/L, and
wherein the second concentration is higher than the first concentration.

11. The p-aminobenzoic acid-producing microorganism as claimed in claim 10, wherein the second sulfonamide antibiotic comprises at least one of the following antibiotics:
sulfanilamide, sulfacetamide, sulfadiazine, sulfasalazine, sulfadimidine and sulfafurazole.

12. The p-aminobenzoic acid-producing microorganism as claimed in claim 10, wherein the first sulfonamide antibiotic and the second sulfonamide antibiotic are the same.

13. The p-aminobenzoic acid-producing microorganism as claimed in claim 10, wherein both of the first sulfonamide antibiotic and the second sulfonamide antibiotic are sulfanilamide.

14. The p-aminobenzoic acid-producing microorganism as claimed in claim 10, wherein the at least one second acclimatized microorganism is the at least one acclimatized microorganism.

15. A p-aminobenzoic acid-producing microorganism derived from a source microorganism,
wherein compared to the source microorganism, the p-aminobenzoic acid-producing microorganism has:
overexpression of a gene related to citrate and malate biosynthesis; and/or
suppressive expression of a gene related to glutamine metabolism, and
wherein the at least one p-aminobenzoic acid-producing microorganism has a higher p-aminobenzoic acid titer than the source microorganism.

16. The p-aminobenzoic acid-producing microorganism as claimed in claim 15, wherein the gene related to citrate and malate biosynthesis comprises at least one of the following genes:
acnA, icd and glcB.

17. The p-aminobenzoic acid-producing microorganism as claimed in claim 15, wherein the gene related to glutamine metabolism comprises at least one of the following genes:
glsA, purF and pyrB.

18. The p-aminobenzoic acid-producing microorganism as claimed in claim 15, wherein the source microorganism comprises Escherichia coli, Corynebacterium glutamicum, Bacillus subtilis, Pseudomonas putida, Yarrowia lipolytica, Saccharomyces cerevisiae or Pichia pastoris.

19. The p-aminobenzoic acid-producing microorganism as claimed in claim 15, wherein the source microorganism is genetically modified Escherichia coli, and the genetically modified Escherichia coli comprises at least one exogenous gene, and the at least one exogenous gene comprises pabA, pabB and pabC.

20. The p-aminobenzoic acid-producing microorganism as claimed in claim 19, wherein compared to the source microorganism, the p-aminobenzoic acid-producing microorganism comprises:
overexpression of a gene related to citrate and malate biosynthesis; and
suppressive expression of a gene related to glutamine metabolism, and
wherein the gene related to citrate and malate biosynthesis comprises acnA, icd and glcB, and the gene related to glutamine metabolism comprises glsA, purF and pyrB.

21. A p-aminobenzoic acid-producing Escherichia coli, which is deposited at Leibniz Institute DSMZ-German Collection of Microorganisms and Cell Cultures and whose deposit number is DSM 34363.


SEQUENCE LISTING (12 sequences)

SEQ ID NO: 1 - DNA, 564 bp, Escherichia coli
  source 1..564: mol_type = genomic DNA
atgatcctgcttatagataactacgattcttttacctggaacctctaccagtacttttgtgaactgggggcggatgtgctggttaagcgcaacgatgcgttgacgctggcggatatcgacgcccttaaaccacaaaaaattgtcatctcacctggcccctgtacgccagatgaagccgggatctcccttgacgttattcgccactatgccgggcgcttgccgattcttggcgtctgcctcggtcatcaggcaatggcgcaggcatttggcggtaaagttgtgcgcgccgcaaaggtcatgcacggcaaaacctcgccgattacacataacggtgagggcgtatttcgggggctggcaaatccacttaccgtgacacgctaccattcgctggtggtggaacctgactcattaccagcgtgctttgacgtgacggcctggagcgaaacccgagagattatggggattcgccatcgccagtgggatctggaaggtgtgcagttccatccagaaagtattcttagcgaacaaggacatcaactgctggctaatttcctgcatcgctga

SEQ ID NO: 2 - protein, 187 aa, Escherichia coli
MILLIDNYDSFTWNLYQYFCELGADVLVKRNDALTLADIDALKPQKIVISPGPCTPDEAGISLDVIRHYAGRLPILGVCLGHQAMAQAFGGKVVRAAKVMHGKTSPITHNGEGVFRGLANPLTVTRYHSLVVEPDSLPACFDVTAWSETREIMGIRHRQWDLEGVQFHPESILSEQGHQLLANFLHR

SEQ ID NO: 3 - DNA, 1362 bp, Escherichia coli
  source 1..1362: mol_type = genomic DNA
atgaagacgttatctcccgctgtgattactttactctggcgtcaggacgccgctgaattttatttctcccgcttaagccacctgccgtgggcgatgcttttacactccggctatgccgatcatccgtatagccgctttgatattgtggtcgccgagccgatttgcactttaaccactttcggtaaagaaaccgttgttagtgaaagcgaaaaacgcacaacgaccactgatgacccgctacaggtgctccagcaggtgctggatcgcgcagacattcgcccaacgcataacgaagatttgccatttcagggcggcgcactggggttgtttggctacgatctgggccgccgttttgagtcactgccagaaattgcggaacaagatatcgttctgccggatatggcagtgggtatctacgattgggcgctcattgtcgaccaccagcgtcatacagtttctttgctgagtcataatgatgtcaatgcccgtcgggcctggctggaaagccagcaattctcgccgcaggaagatttcacgctcacttccgactggcaatccaatatgacccgcgagcagtacggcgaaaaatttcgccaggtacaggaatatctgcacagcggtgattgctatcaggtgaatctcgcccaacgttttcatgcgacctattctggcgatgaatggcaggcattccttcagcttaatcaggccaaccgcgcgccatttagcgcttttttacgtcttgaacagggtgcaattttaagcctttcgccagagcggtttattctttgtgataatagtgaaatccagacccgcccgattaaaggcacgctaccacgcctgcccgatcctcaggaagatagcaaacaagcagtaaaactggcgaactcagcgaaagatcgtgccgaaaatctgatgattgtcgatttaatgcgtaatgatatcggtcgtgttgccgtagcaggttcggtaaaagtaccagagctgttcgtggtggaacccttccctgccgtgcatcatctggtcagcaccataacggcgcaactaccagaacagttacacgccagcgatctgctgcgcgcagcttttcctggtggctcaataaccggggctccgaaagtacgggctatggaaattatcgacgaactggaaccgcagcgacgcaatgcctggtgcggcagcattggctatttgagcttttgcggcaacatggataccagtattactatccgcacgctgactgccattaacggacaaattttctgctctgcgggcggtggaattgtcgccgatagccaggaagaagcggaatatcaggaaacttttgataaagttaatcgtatcctgaagcaactggagaagtaa

SEQ ID NO: 4 - protein, 453 aa, Escherichia coli
MKTLSPAVITLLWRQDAAEFYFSRLSHLPWAMLLHSGYADHPYSRFDIVVAEPICTLTTFGKETVVSESEKRTTTTDDPLQVLQQVLDRADIRPTHNEDLPFQGGALGLFGYDLGRRFESLPEIAEQDIVLPDMAVGIYDWALIVDHQRHTVSLLSHNDVNARRAWLESQQFSPQEDFTLTSDWQSNMTREQYGEKFRQVQEYLHSGDCYQVNLAQRFHATYSGDEWQAFLQLNQANRAPFSAFLRLEQGAILSLSPERFILCDNSEIQTRPIKGTLPRLPDPQEDSKQAVKLANSAKDRAENLMIVDLMRNDIGRVAVAGSVKVPELFVVEPFPAVHHLVSTITAQLPEQLHASDLLRAAFPGGSITGAPKVRAMEIIDELEPQRRNAWCGSIGYLSFCGNMDTSITIRTLTAINGQIFCSAGGGIVADSQEEAEYQETFDKVNRILKQLEK

SEQ ID NO: 5 - DNA, 810 bp, Escherichia coli
  source 1..810: mol_type = genomic DNA
atgttcttaattaacggtcataagcaggaatcgctggcagtaagcgatcgggcaacgcagtttggtgatggttgttttaccaccgccagagttatcgacggtaaagtcagtttgttatcggcgcatatccagcgactacaggatgcttgtcagcggttgatgatttcctgtgacttctggcctcagcttgaacaagagatgaaaacgctggcagcagaacagcaaaatggtgtgctgaaagtcgtgatcagtcgcggtagtggcgggcgagggtacagcacattgaacagcggaccggcaacgcggattctctccgttacggcttatcctgcacattacgaccgtttgcgtaacgaggggattacgttggcgctaagcccggtgcggctggggcgcaatcctcatcttgcaggtattaaacatctcaatcgtcttgagcaagtattgattcgctctcatcttgagcagacaaacgctgatgaggcgctggtccttgacagcgaagggtgggttacggaatgctgtgcggctaatttgttctggcggaagggcaacgtagtttatacgccgcgactggatcaggcaggtgttaacggcattatgcgacaattctgtatccgtttgctggcacaatcctcttatcagcttgtcgaagtgcaagcctctctggaagagtcgttgcaggcagatgagatggttatttgtaatgcgttaatgccagtgatgcccgtatgtgcctgtggcgatgtctccttttcgtcagcaacgttatatgaatatttagccccactttgtgagcgcccgaattag

SEQ ID NO: 6 - protein, 269 aa, Escherichia coli [sequence omitted]

SEQ ID NO: 7 - DNA, 60 bp, synthetic construct
  source 1..60: mol_type = other DNA
ggacgcactgaccgaattcattaaagaggagaaaggtaccatgatcctgcttatagataa

SEQ ID NO: 8 - DNA, 60 bp, synthetic construct
  source 1..60: mol_type = other DNA
ggaattcgatatcaagcttatcgataccgtcgacctcgagtcagcgatgcaggaaattag

SEQ ID NO: 9 - DNA, 60 bp, synthetic construct
  source 1..60: mol_type = other DNA
ggacgcactgaccgaattcattaaagaggagaaaggtaccatgaagacgttatctcccgc

SEQ ID NO: 10 - DNA, 60 bp, synthetic construct
  source 1..60: mol_type = other DNA
ggaattcgatatcaagcttatcgataccgtcgacctcgagttacttctccagttgcttca

SEQ ID NO: 11 - DNA, 60 bp, synthetic construct
  source 1..60: mol_type = other DNA
ggacgcactgaccgaattcattaaagaggagaaaggtaccatgttcttaattaacggtca

SEQ ID NO: 12 - DNA, 60 bp, synthetic construct
  source 1..60: mol_type = other DNA
ggaattcgatatcaagcttatcgataccgtcgacctcgagctaattcgggcgctcacaaa